(19) United States
(12) Patent Application Publication
(10) Pub. No.: US 2020/0305938 A1
(43) Pub. Date: Oct. 1, 2020

(54) DYNAMIC COMPRESSION FIXATION DEVICES

(71) Applicant: Helix Fixation, Inc., Pleasanton, CA (US)

(72) Inventors: John F. KRUMME, Bainbridge Island, WA (US); Karl KRUMME, Bainbridge Island, WA (US)

(21) Appl. No.: 16/831,528

(22) Filed: Mar. 26, 2020

Related U.S. Application Data
(60) Provisional application No. 62/824,311, filed on Mar. 27, 2019.

(51) Int. Cl. A61B 17/72; A61B 17/86

(52) U.S. Cl. CPC A61B 17/7225; A61B 17/869; A61B 2017/00867; A61B 17/8635; A61B 17/864

(57) ABSTRACT

Devices and methods are disclosed for orthopedic uses, such as treating and compressing a broken bone. An implantable device may be provided with an elongate body, a head region, a bone engagement part such as an anchor region or threads, and a dynamic compression portion in either a first axially compact configuration or a second axially elongated configuration and configured to transform between the first axially compact configuration and the second axially elongated configuration.

### CROSS REFERENCE TO RELATED APPLICATIONS

This application claims priority to U.S. Provisional Patent Application No. 62/824,311 filed Mar. 27, 2019 and titled “Dynamic Compression Fixation Devices” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure is related to implantable devices, especially implantable devices for orthopedic uses. In particular this disclosure is related to implantable devices that may provide dynamic compression to a broken bone.

### BACKGROUND

The skeletal system of the body includes 206 bones and numerous joints. The bones and joints act as a scaffold by providing support and protection for the soft tissues. The bones and joints are also necessary for body movement. The bones of the skeletal system are made up of calcium and minerals, as well as cells and proteins. Bones can be broken due to trauma or force, such as falling on a sidewalk or being hit by a car. Various physiological processes in the body act to heal broken bones. Starting from soft, inflamed, swollen tissue at the bone fracture site, the body's natural process of healing a bone fracture includes a healing progression that takes place over the course of a number of weeks or months. After an initial inflammation, the process moves to repairing the damage, and finally moves on to remodeling the bone. The average time to heal a broken bone is between 6-8 weeks (and longer if full reshaping is considered), although the actual amount of time varies depending on a number of factors specific to the injury, including type of injury, the site of the injury, the grade of the injury, other tissue damage, and the age and health of the patient. Although a continuous process, average bone fracture healing can be divided into a number of stages based on the physiological healing process, including inflammation and hematoma formation (0-2 weeks), soft callus formation (2-3 weeks), hard callus formation (3-6 weeks), and bone remodeling (8 weeks-2 years).

Common practices in bone fracture treatments include providing compression to and stabilization of the broken bone. Some bone fracture treatments include non-surgical approaches, such as using splints to minimize movement, braces to support the bone, or casts to support and immobilize the bone. Some bone fracture treatments are surgical and involve surgically inserting implants in or around the bones. For treating some bone fractures, special screws are placed in the broken bone to hold the broken pieces close together. For treating some bone fractures, such as fractures of the thigh bone or shin bone, a special plate called a bone plate may be placed on the fractured bone segments to stabilize, protect, and align the fractured bone segments for healing. The bone plate can be held on the bone with screws that screw into the bone. To stabilize some fractures, a long rod, called an intramedullary rod, may be placed inside the bone. The intramedullary rod may be held inside the bone using screws screwed through the rod and the bone.

According to the American Academy of Orthopaedic Surgeons, an average of more than 6 million people in the United States break a bone every year. Although many of these broken bones heal properly, many others do not. It is estimated that of those broken bones, up to 20% will not heal properly. Improper healing includes delayed union (the fractured bone takes longer than usual to heal), malunion (the fractured bone heals in an abnormal position), and nonunion (the fracture does not heal). A number of factors can contribute to improper healing, and it is generally thought that bone misalignment during the weeks-long healing process is a major contributor to improper healing. Improper healing of broken bones can result in loss of function, decreased quality of life, swelling, chronic pain, inability to work, limited ability to work or recreate, and additional medical and hospital costs.

Thus there is a need for improved devices and methods to improve outcomes for patients with broken bones. Described herein are apparatuses and methods that may address these and other problems.

### SUMMARY OF THE DISCLOSURE

The present disclosure relates to apparatuses (devices and methods) for orthopedic uses. In particular this disclosure is related to implantable devices that may provide dynamic compression to a broken bone.

One aspect of the disclosure provides an implantable device including an elongate body with a proximal end and a distal end; a head region at the proximal end wherein the head region is wider than other portions of the elongate body; a bone engagement part at the distal end configured to engage a bone; and a dynamic compression portion between the head region and the bone engagement part, the dynamic compression portion in either (i) a first axially compact configuration or (ii) a second axially elongated configuration, the dynamic compression portion comprising a material configured to transform between the first, compact configuration and the second elongated configuration.

In some embodiments, the second, elongated configuration is at least 0.5% longer than the first, compact configuration.

In some embodiments, the dynamic compression portion includes nitinol. In some embodiments, the dynamic compression portion includes a cannulated rod.

In some embodiments, the dynamic compression portion comprises a cannulated rod with a rod wall and a helical slit through a wall thickness thereof. In some embodiments, the dynamic compression portion includes a helix having at least two, at least three, at least four, at least five, or at least six helical turns.

In some embodiments, when the dynamic compression portion is in the second configuration, the dynamic compression portion urges the proximal end and the distal end towards each other. In some embodiments, the second, elongated configuration is at least 1%, at least 2%, at least 5% or at least 10% longer than the first, compact configuration.

In some embodiments, the bone engagement part comprises a screw thread. In some embodiments, the bone engagement part includes an anchor or tab. In some embodiments, the dynamic compression portion includes a helix and the screw thread and the helix turn in the same direction. In some embodiments, the dynamic compression portion comprises a helix and the screw thread and the helix turn in opposite directions. In some embodiments, the bone engagement part includes a helical thread.

In some embodiments, the bone engagement part includes a helical thread with either a first configuration with a smaller transverse width or a second configuration with a larger transverse width, wherein the helical thread is configured to transform between the first configuration and the second configuration in response to an applied or removed force. In some embodiments, the bone engagement part includes an anchor, such as an extendible tab. In some embodiments, the bone engagement part has an outer diameter greater than an outer diameter of the dynamic compression portion. In some embodiments, the head region has an outer diameter greater than an outer diameter of the dynamic compression portion.

Another aspect of the disclosure provides a method of securing bone segments together including the step of introducing an implantable device through a first bone segment and at least partially into a second bone segment, the implantable device having: an elongate body with a proximal end and a distal end; a head region at the proximal end and wherein the head region engages with a proximal end of the first bone segment; a bone engagement part at the distal end wherein the bone engagement part engages with the second bone segment; a dynamic compression portion between the head region and the bone engagement part, the dynamic compression portion in a first axially compact configuration. Some embodiments include the step of transforming the dynamic compression portion into a second axially elongated configuration. Some embodiments include the step of urging the dynamic compression portion from the second elongated configuration towards the first, compact configuration, thereby urging the first bone segment and the second bone segment together. In some embodiments, the dynamic compression portion axially contracts by at least 0.5% relative to the length of the axially compact configuration. In some embodiments, the head region is wider than other portions of the elongate body.

Some embodiments includes the step of drilling a first channel through the first bone segment and drilling a second channel at least partially through the second bone segment. In some embodiments, the implantable device is configured to axially contract at least 1%, at least 2%, at least 5% or at least 10% relative to the length of the axially compact configuration.

Some embodiments include the step of contracting the implantable device by at least 1%, at least 2%, at least 5% or at least 10% relative to the length of the axially elongated configuration toward the axially compact configuration after the implantable device has been introduced into the bone segments.

Some embodiments include the step of transforming the bone engagement part of the implant from a radially smaller structure to a radially larger structure and thereby engaging the second bone segment and holding the implantable device in the second bone segment.

In some embodiments, the dynamic compression portion includes a hollow region having a helical slit through a wall thickness thereof. In some embodiments, urging includes axially compacting the helical slit.

In some embodiments, a diameter of the dynamic compression portion remains relatively constant during the urging step.

In some embodiments, the dynamic compression portion and bone engagement part comprise Nitinol. In some embodiments, the head region remains outside the first bone segment. In some embodiments, the introducing step requires substantially zero insertion force.

### BRIEF DESCRIPTION OF THE DRAWINGS

The novel features of the invention are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1A shows a prior art bone screw with limited compression.

FIG. 1B shows a stress-strain graph comparing the properties of the device shown in FIG. 1A with properties of a typical bone.

FIG. 2A shows an implantable device with dynamic compression and improved material properties.

FIG. 2B shows a stress-strain graph showing the overlap in material properties of the device shown in FIG. 2A with properties of a typical bone.

FIG. 2C shows the implantable device of FIG. 2A in place across a break in a bone.

FIG. 3A shows two configurations of implantable device with a helical region for dynamic compression and improved material properties.

FIG. 3B shows a stress-strain graph showing the overlap in material properties of the device shown in FIG. 3A with properties of a typical bone.

FIG. 4A shows two configurations of implantable device made from Nitinol with a proximal helical region for dynamic compression and a compressible distal threaded region.

FIG. 4B shows the implantable device shown in FIG. 4A after implantation.

FIG. 4C shows a stress-strain graph showing the overlap in material properties of the device shown in FIGS. 4A-4B with properties of a typical bone.

FIG. 5A shows two configurations of implantable device made from a titanium alloy or stainless steel with a proximal helical region for dynamic compression and a distal threaded region.

FIG. 5B shows a stress-strain graph showing the overlap in material properties of the device shown in FIG. 5A with properties of a typical bone.

FIG. 6A shows examples of an implantable device made from a titanium alloy or stainless steel distal anchor region.

FIG. 6B and FIG. 6C are two enlarged fragmentary views showing the distal anchor region of one of the devices from FIG. 6A, shown first with the anchors being held in a retracted state by an inserting tool (FIG. 6B), and then in a deployed state with the tool removed (FIG. 6C).

FIG. 6D shows examples of an implantable device with a proximal helix and a compressible distal threaded region.

FIG. 6E and FIG. 6F are two enlarged fragmentary views showing the distal anchor region of one of the devices from FIG. 6C, shown first with helical threads in a retracted state (FIG. 6E) and then in a deployed state (FIG. 6F).

FIG. 7A shows examples of an implantable device with a proximal helix and a distal threaded region, such as for a surgical kit.

FIG. 7B shows examples of an implantable device with a proximal elastic region and a distal threaded region, such as for a surgical kit.

FIG. 7C shows examples of an implantable device with a proximal helical region and a distal threaded region, such as for a surgical kit.

FIG. 7D shows examples of an implantable device with a proximal longitudinal slot and a distal threaded region, such as for a surgical kit.

### DETAILED DESCRIPTION

Described herein are apparatuses and methods for orthopedic uses. In particular, described herein are implantable devices that may be especially useful for treating, repairing, or supporting a broken or damaged bone. The implantable devices may be useful for reducing bone fractures to provide proper compression and stabilization to a broken bone joint to support joint regrowth, healing and mechanical support during the healing process. The implantable devices may also be useful for fixing or holding another bone device, such as a bone plate or intramedullary rod, in place. The disclosure herein provides those functions and enhances bone healing rate and strength of the joint while providing better ease of use and fatigue failure resistance. As indicated above, bone fracture healing takes place in a continuous series of stages. During these stages, repair tissue for repairing the bone fracture progresses from soft tissue to a soft callus to a hard callus and then bone remodeling. The material properties of the tissue changes during these stages. As a bone fracture heals, tissues are resorbed and remodeled. Inflammation reduces. The initial placement of the segments of broken bone may have been appropriate, but needs change over time and the initial placement of the bones may not be ideal over time. Although a broken bone needs to be held in place during healing, very tiny movements referred to micromovements, may aid in recovery. The implantable devices described herein may provide a better match to the material properties of healing bone over time than do existing devices. The implantable devices described herein may provide or be configured to provide appropriate dynamic compression to a fixtured bone initially as well as over an extended period of time (weeks, months, or years), as the bone heals and remodels. The implantable devices disclosed herein may provide controllable, dynamic, continuing compression to a fixtured joint and may lead to enhancing bone regrowth through modulus matched elastic properties (device to bone elastic properties); providing improved fatigue failure resistance; and in some embodiments, eliminating the twisting, screwing action and torque associated with prior art threaded screw devices made from titanium and stainless steel; and allowing micro-motion of the compressed joint for faster and stronger joint healing.

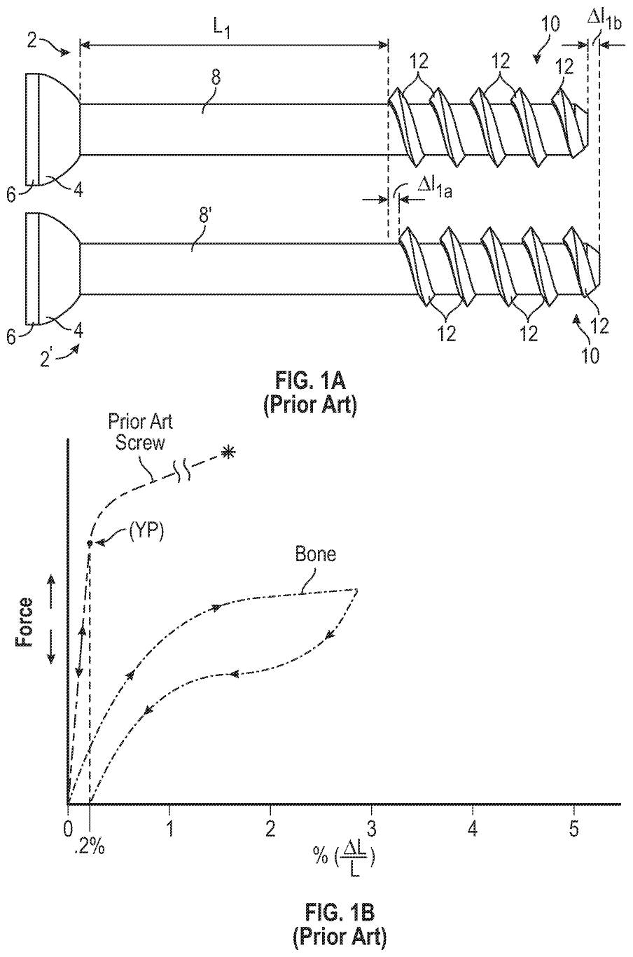

FIG. 1A shows views of a prior art screw 2 (e.g., a prior art “compression” screw) as it would appear before insertion (top) and the screw 2′ after insertion (bottom) across a broken bone to hold or fix the bone into position. The screw 2 has a distal threaded end 6, a smooth surfaced central axial length 8, and a proximal head 4. FIG. 1A shows the smooth surfaced central axial length 8 has a diameter smaller than the diameter of the distal threaded end 10. In use, the screw 2 is inserted into a bone by rotating a screwdriver (not shown) that engages a mating part 6 on the proximal end of the screw and rotates the screw 2 into the broken bone. The head 4 of the screw keeps the screw 2 on the outside of the bone (such as due to its wide size or a head shape that engages directly with the bone or a plate), while torque from the screwdriver causes the screw 2 to rotate into the broken bone, forcing threads 12 on the distal end 10 of the screw 2 into the bone. The screw 2 is made of implant grade titanium and/or stainless steel. As shown in FIG. 1A, as the screw 2 is inserted into the bone (not shown in this view) the torque placed on the screw threads 12 stretches the central zone 8 by a small amount ΔL1a to central zone 8′ and the tip of the screw 2′ extends further into the bone by the same distance, shown by ΔL1b. The axial central zone can be solid or tubular, but has no geometry that enhances the elastic properties of the device overall. As a result, the extent of any “compression” performance of the screw 2′ is limited to the inherent low strain yield point (YP) of the titanium and/or stainless steel at 0.2%. FIG. 1B compares the stress-strain curve of force vs. % elongation for the titanium screw 2 shown in FIG. 1A with elastic properties of a typical human bone. A stainless steel screw behaves similarly to the titanium screw. Note that the “range of motion” is just 0.2% and the steep slope of the titanium/stainless steel screw does not overlay or match the bone properties graph. The initial small amount of compression provided is lost within minutes after installation. Any further rotation of the screw simply results in bone movement. At stress below the yield point (YP), the screw 2′ shows elastic behavior and if stretched can return to its original size and shape. When inserted with a force below the yield point, the screw can exert compressive stress between two parts of a broken bone as it is pulled towards its original size and shape. However, if the screw is stretched beyond its yield point, the screw 2′ shows permanent strain deformation (plastic behavior); it has been stretched too far and cannot return to its original size. It cannot exert a desired compressive force on the healing bone. FIG. 1B also shows the significant mismatch between the elastic properties of a typical bone and a titanium (or stainless steel) screw. Where there is a mismatch, stress on the bone is increased; the greater the mismatch, the greater the stress.

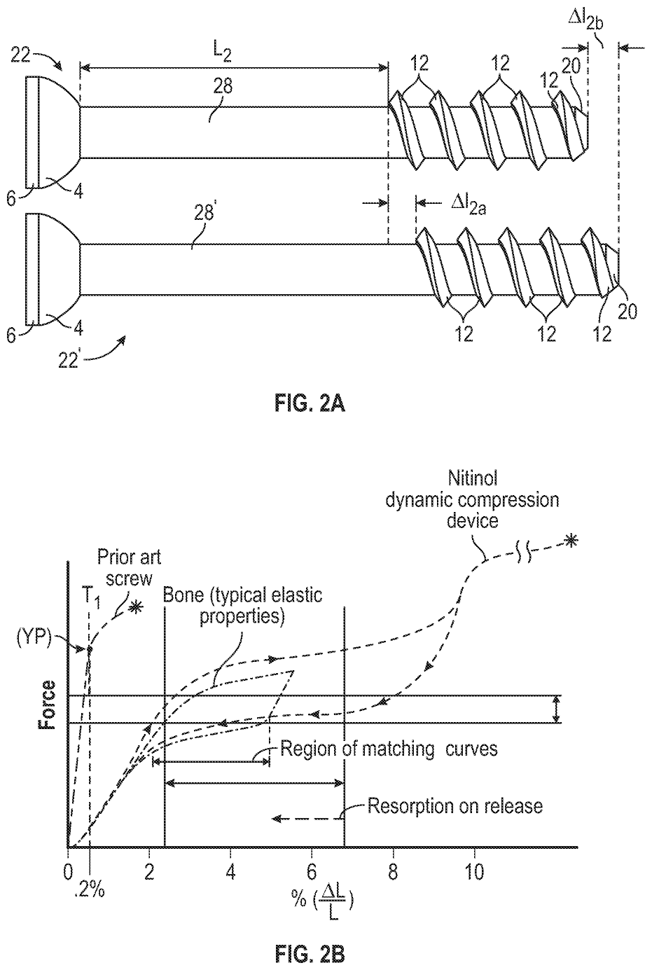

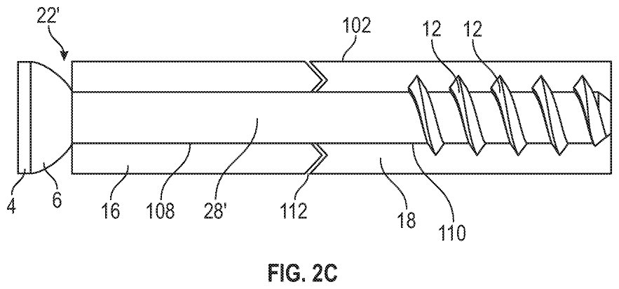

Described herein are implantable devices, such as bone screws, with improved material properties. For example, the implantable devices may have spring-like geometry in an axial central zone and/or elastic properties closer to that of bone compared with existing devices. The implantable devices may have an elongate body with a proximal end and a distal end and a dynamic compression region (e.g., an elastic central zone) between the proximal end and the distal end. The central zone may be unthreaded and of a suitable length for the specific joint size and depth. The central zone may move between a first, axially compact configuration and a second, axially elongated configuration. The central zone may be made from an elastic material and can be considered to have “elastic stretch”. FIG. 2A shows an implantable device 22 configured to deliver dynamic compression. The implantable device 22 has a head 4 at the proximal end and a distal helically threaded zone with threads 12 at the distal end 20, and a central zone 28 (e.g., a dynamic compression portion) between the head 6 and the threads 12. The central zone may provide spring like geometry that enhances the elastic behavior. The central zone 28 also uses an elastic material (e.g., Nitinol) rather than titanium or stainless steel. Nitinol has a Yield Point (stress/strain point resulting in permanent deformation) at approximately 40 times greater strain than that of the titanium and stainless steel. The Nitinol may be particularly useful for its properties of elasticity, strength, and biocompatibility, rather than as a thermal controlled shape memory material; the thermal shape memory function does not need to be utilized for this purpose. The central zone 28 may be configured to have a first axially compact configuration central zone 28 and a second axially elongated configuration central zone 28′, and the central zone 28 may transform smoothly or continuously between these configurations. In some examples, the central zone 28 may be placed into the second, axially elongated configuration as the implantable device is screwed into a bone channel. FIG. 2C shows the implantable device 22′ in place in a broken bone 102. The implantable device 22 can be screwed into pre-drilled bone channels of a broken bone to be joined together with compression. As the implantable device 22 is screwed into place in the broken bone 102, the distal helically threaded zone with threads 12 at the distal end 20 are positioned across the break (joint) entirely into the second bone segment 106 on the distal or far side bone. As the implantable device 22 is further advanced/screwed in, the proximal head 4 will engage the first bone segment 16 on the near side. Further rotation of the screw will pull the far side against the near side of the joint compressing the joint. The compression force increases with further rotation until the plateau stress level in the central zone of the Nitinol device is reached, and from that point on, further rotation of the implantable device 22 will result in approximately constant compression of the joint up to about 6% of the length of the central zone 8. This compression range (6% plus typical for NiTi I SE 508 SR) and the “close to constant” force attributes of the Nitinol plateau strain behavior allow a large degree of latitude for the surgeon to bring the bone joint segments together, apply a compression level (which may be pre-determined), and to accommodate the immediate joint resorption while maintaining compression over time. Devices made from titanium or stainless steel seldom can retain any compression on a joint even for a short time after insertion since resorption of the bone surfaces in compression is often greater than the small compression motion the 0.2% strain yield point of these materials provides. The central zone 28 may be lengthened (deformed) into the second, axially elongated configuration. The central zone 28 may also be lengthened into the second, axially elongated configuration and held. In some variations, the central zone 28 may be cannulated (tubular/hollow). In some other variations, the central zone is non-cannulated (solid).

In order to axially extend and/or axially contract, the central zone of an implantable compression screw as described herein may be configured to move (e.g., glide or slide) through a bone channel through which it is inserted. For example, the central zone may be relatively smooth (unthreaded), although the distal end may be threaded. The central zone may lack other features (e.g., anchor tabs) that may be present at the distal end of the implantable device for anchoring the implant relative to a bone channel. The central zone 28 may be a suitable length for a specific joint size and depth into a bone. A head 4 of a screw may have a mating part 6 for mating with a screwdriver (e.g., a hexagonal, Allen, torq, slotted, cruciate, or Philips screwdriver; not shown). FIG. 2B shows a comparison of material properties for an implantable device 22 (made of Nitinol) as described herein and another typical bone (such as a cortical bone). FIG. 2B shows a stress-strain graph of force vs. % elongation. The curve for the titanium screw shown in FIG. 1A is shown at the left for reference. When inserted at a stress below the yield point, the implantable device 22 can exert compressive stress on the broken bone as the bone is compressed or pulled towards its original size and shape. However, if the implantable device 22 is stretched beyond the yield point, the implantable device 22 shows permanent strain deformation (plastic behavior); it has been stretched too far and cannot return to its original size. Thus, beyond this point it cannot exert a desired compressive force on the healing bone. The device curve closely matches the human bone curve over a range of motion of greater that 3% at close to constant force. Further rotation/screwing-in of the device does not increase the compression level and provides a total range of motion over 6% to accommodate resorption and any alignment associated motion as well.

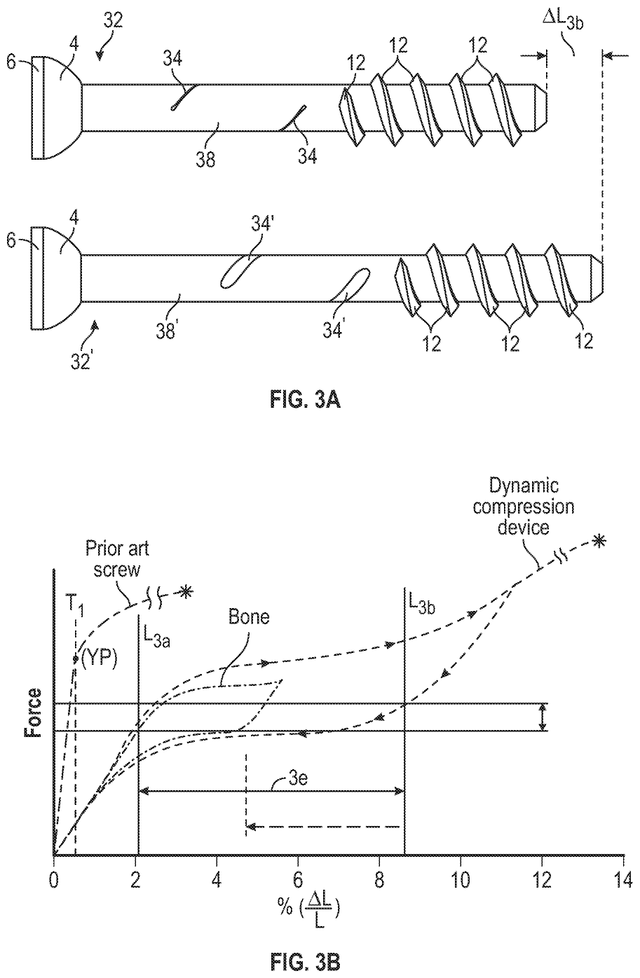

Also described herein are implants that include a feature, such as a geometrical or mechanical feature, and the feature may control (at least in part) feature elasticity, feature compression, and/or feature length. The feature may be in a central zone of an implant and changes to the feature may control and change device elasticity, device compression, and/or device length. In some examples, a feature may be a helical region or part of a helical region in the central zone of an implantable device. A feature, such as a helix (as used herein, a helix also includes a spiral and variations) may have spring or spring-like properties. A central zone (or an entire implant) may be made from a material that itself has good elastic characteristics (e.g., greater than the elasticity of stainless steel or titanium implant material), such as Nitinol. The central zone having the feature may be cannulated (hollow) or non-cannulated (e.g., a solid rod). FIG. 3A shows an example of an implantable device 32 (a dynamic compression screw) in an unstressed state. Similar as to described above for the implantable device 22, implantable device 32 has head region 4 at the proximal end and a distal helically threaded zone with threads 12 at the distal end 20. Implantable device 32 has a central zone 38 between the head region 6 and the threads 12, and the central zone 38 includes a helix 34. Similar as to described above for the implantable device 22, the implantable device 32 can be inserted into a bone using a screwdriver that mates with mating part 6 at the proximal end. The head region 4 can hold the proximal end of the implantable device 32 on the proximal end of a first bone segment, the threads 12 can anchor the implantable device 32 into a second bone segment on a distal end, and the central zone 38 can cross a break in the bone. Rotating/screwing implantable device 32 further into the bone, the break in the bone can be reduced, and the proximal segment and distal segment of the bone can be pulled closer together. As shown in the bottom of FIG. 3A, rotating/screwing implantable device 32 into the bone, elongates helix 34′, the central zone 38′, and implantable device 32′ by a length represented by AL3b. The helix 34/34′ may be configured to axially elongate and axially contract, increasing and decreasing the length of the central zone 38/38′ and the implantable device 32/32′. The helix 34/34′ may be configured to axially elongate and axially contract in response to applied force, and may do so continuously rather than “snapping” from the top configuration to the bottom configuration. Once elongated, the elongated helix 34′ can provide compressive force over the bone segments during bone healing (e.g., for 0-8 weeks such as for normal bone healing, or longer (e.g., for 8 weeks-2 years, or longer for abnormal bone healing or additional bone remodeling).

FIG. 3B shows a stress-strain graph of elastic properties of the implantable device 32/32′ shown in FIG. 3A. The graph of elastic properties in FIG. 3B shows the effect of adding a helical cut to the central length of a Nitinol device (such as the one shown in FIG. 2A), creating a spring like elastic action in this zone as the helical cut is stretched and released (see the dotted line with right pointing and left pointing arrowheads showing, respectively, elastic stretch and elastic recovery). FIG. 3B shows that a total range of motion for the implantable device 32/32′ can be greater than 12%, with no permanent strain. The elastic action can be controlled by various parameters (e.g., adjusting the pitch or number of turns of the spiral per unit length) and the wall thickness of tubular body of the device. FIG. 3B also shows that matching implant properties to bone properties with constant compression forces, greater than a 3% range of motion is achieved. This range of compressive motion is greatly increased over the range compared to the prior art screw shown on the left side of the figure and in FIG. 1B. FIG. 3B also shows the implant range of axial motion or working compression e (between L1 and L2) is from about 2% to about 8.5% elongation/contraction. FIG. 3B also shows the degree f of implant contraction upon implantation due to resorption upon release. A feature may be a helix and/or a spiral extending or wrapping around a longitudinal axis of an implantable device, or may be another geometrical/mechanical feature or form (e.g., a series of holes or slits) that improves elasticity especially in the central zone of an implant. A helix may be generally circular (e.g., have a generally constant radius or constant curvature). A spiral may include winding in continuous and gradually widening or tightening manner, such as in a curve around a longitudinal axis of the implant. A helix or spiral may be regular or irregular. A helix or spiral may be curved, bulging, flattened, or rounded. In some variations, a helix or spiral may be circular, ovoid, triangular, saddled, or square. A feature, such as a helix or spiral, may be made using a laser cut into a rod or by winding and shaping a wire to the desired shape. Various parameters of a feature, such as a helix wall thickness of an elastic (e.g., Nitinol) tube in the central elastic zone, the pitch of the helix, the number of turns of the helical cut per unit length, and/or the number of helices control enable the elastic behavior of the device. The device can be pre-designed allowing control of range of motion and absolute compression forces of the device. A thicker tube wall can result in higher absolute forces, while retaining the range of motion. Higher pitch (turns/length) can result in larger range of motion at the expense of absolute forces. A wall thickness of a helix can be greater than 0.1 mm, greater than 0.5 mm, greater than 1 mm, greater than 2 mm, greater than 5 mm or less than 5 mm, less than 2 mm, less than 1 mm, less than 0.5 mm or and values between these. A wall thickness can extend all the way to an implant hollow interior, or can extend partway. A pitch of a helix can be a desired size such as greater than 0.1 mm, greater than 0.5 mm, greater than 1 mm, greater than 2 mm, greater than 5 mm or less than 5 mm, less than 2 mm, less than 1 mm, less than 0.5 mm or and values between these. A feature, such as a helix, can have at least one turn, at least two turns, at least three turns, at least four turns, at least five turns or at least ten turns, or not more than ten turns, not more than five turns, not more than four turns, not more than three turns, not more than two turns or one turn or fewer than one turn or values between these (e.g., at least one turn and not more than six turns). Although the number of helical turns, can be an integer (1, 2, 3, etc.,) the number of turns can also be fractional (e.g., two and a half turns). In some variations, a feature (a helix) may be a partial helix (e.g., half of a helix). An implant may include one helical feature (one helix) or more than one helix. For example, two helices could be wrapped around a single length of a central zone, or two helices could be placed longitudinally adjacent to each other. This added geometry to the elastic central zone of the device allows the range of motion to be extended from about 6% (for a Nitinol central zone without this geometry) to beyond 15% (for a Nitinol central zone with this geometry), thus giving a surgeon more latitude for adjustment and less sensitivity to particular positioning of the device. The flexibility of the device introduced by the feature or helix also contributes to more fatigue failure resistance since small deflections are accommodated and recovered. A subset of any different implant features or parameters listed herein can be combined and may be particular suited to a certain bones or groups of bones (e.g., thigh bone (femur), kneecap (patella), shin bone (tibia), fibula, shoulder blade (scapula), collar bone (clavicle), humerus, radius, ulna, cervical, thoracic and lumbar vertebrae, sacrum, tailbone (coccyx), skull, jawbone, ribs, breastbone (sternum), wrist bones (carpals), metacarpals and phalanges. In some variations, part of an implant as described herein may cross an intervening structure, such as a bone plate and may hold the intervening structure in place, against a bone.

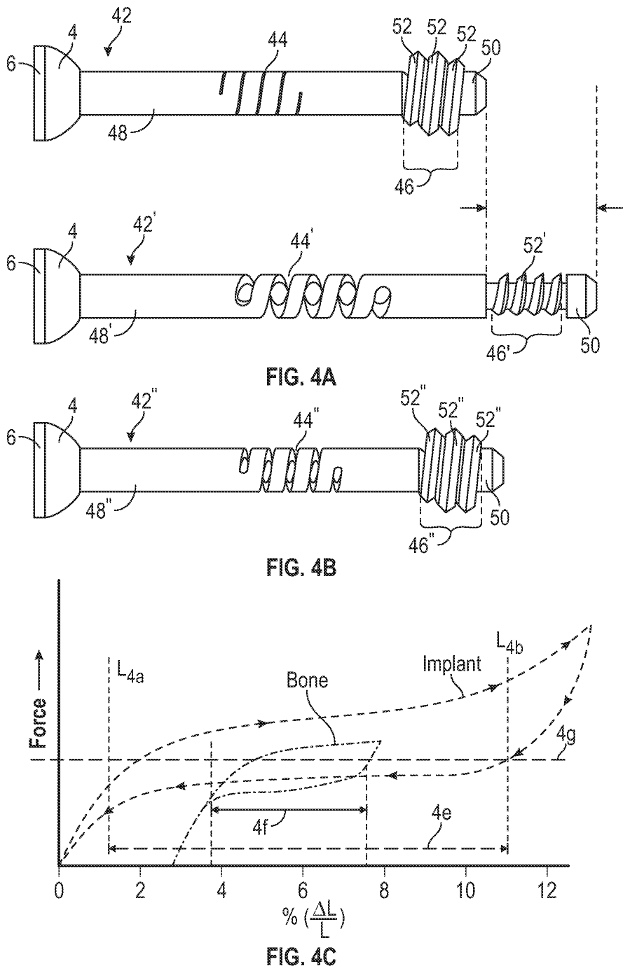

Also described herein are implantable devices requiring zero insertion force (ZIF). These implantable devices are inserted, in part using a shape memory material and do not require torque, rotation, or tapping for insertion into a broken bone. The implantable devices may have a distal anchor feature that can transform from a radially compressed shape (e.g., a deformed, shape memory shape) for insertion to a radially expanded shape for anchoring the implant in a bone channel. The distal anchor feature may be a helical coil zone and may have a thread geometry in the Nitinol tube distal end that resembles a typical thread geometry. They may be inserted by a different mechanism from that of a typical threaded distal end. FIG. 4A shows an example of an implantable device 42 requiring zero insertion force for inserting into a bone. The implantable device 42 at the top of FIG. 4A shows the device as manufactured from NiTinol before deformation. The implantable device 42 is similar to the implantable device 32 shown in FIG. 3A, except that the distal threaded region 12 in the implantable device 32 shown in FIG. 3A is replaced with an anchor 46 at the distal end 50 of the implantable device 42. The anchor 46 is provided as a helical coil zone resembling a typical thread geometry in the Nitinol tube distal end. Similar to as described above for FIG. 3A, the implantable device 42 may be made of an elastic material, such as Nitinol. However, here the anchor 46 may be “shape set” using Nitinol shape setting techniques. FIG. 4A also shows the implantable device 42′ deformed, and axially pre-stretched by about 6% as shown by the double dotted lines comparing the length of the deformed implantable device 42′ with the implantable device 42 as manufactured. The implantable device 42′ is ready for insertion in a pre-drilled hole in a bone needing fixation. The anchor 46 can be diametrically expanded to anchor 46′ as shown in FIG. 4A (e.g., at least 1.1× its original diameter, at least 1.5× its original diameter, at least 2.0× its original diameter, at least 2.5× its original diameter, or not more than 2.5× its original diameter, not more than 2.0× its original diameter not more than 1.5× its original diameter or any value between these amounts (e.g., at least 1.5× and not more than 2.3× its original diameter)). For insertion into a bone channel in a bone, expanded anchor 46 of the implantable device 42 shown in the top of FIG. 4A is compressed back to its original anchor 46′ size, using an insertion tool (not shown in this view), such as a coaxial installer configured to extend inside a hollow implantable device 42. The insertion tool retains the anchor 46′ of the implantable device 42′ in the compressed state ready for installation in a pre-drilled bone channel. The insertion tool also axially stretches the implantable device 42′ (e.g., the central zone 44′) prior to insertion of the implantable device 42′ into a bone channel. In some examples, a first bone segment is separated from a second bone segment by a bone break, and a first channel is made through the first bone segment and a second channel is made at least partially through the second bone segment, such as by drilling with a bone drill. The implantable device 42′ can be inserted into a bone using the insertion tool. The implantable device 42′ can be inserted through the first channel, across the bone break, and into the second channel to a desired depth. The head region 4 keeps the proximal end of the implantable device 42′ on the proximal end of the first bone segment. The insertion tool is actuated, releasing the diametrically compressed anchor 46′ at the distal end. FIG. 4B shows the implantable device 42′ as recovered upon release from the insertion tool. The anchor 4 has transformed to a screw-like shaped anchor 46″ and the axial pre-stretch in length has also mostly recovered. The diametrically compressed anchor 46 expands into the bone (e.g., bone surrounding the second bone channel) like a screw. The accessory tool is withdrawn from the implantable device 42″ releasing the axial pre-stretched central zone 48′ which shortens to central zone 44″ (as seen in FIG. 4B), compressing the joint (break). The joint may be compressed to a pre-designed compression level. The central zone 48″ may axially shorten (relative to the insertion configuration shown in the bottom of FIG. 4A). The joint may be compressed to a pre-designed compression level. The central zone 48″ (and the helix 44″) are elongated relative to the starting configuration of the central zone 48 (and the helix 44) shown in the implantable device 42 at the top of FIG. 4A. This double Nitinol super elastic recovery action will both anchor the anchor 46″ in the distal bone segment and compress the distal segment and proximal segment axially together with a wide range of motion to accommodate resorption, initial compression of the joint, and continuing compression over time to enhance regrowth and strength of the repair site.

If further compression is desired, the implantable device 42″ can be rotated/further inserted like a screw, such as by mating a screwdriver with mating part 6 on the proximal end of the implantable device and rotating the implantable device 42″ (e.g., rotating clockwise). The head region 4 can hold the proximal end of the implantable device 42″ on the proximal end of a first bone segment and the implantable device 42″ can be rotated so that threads 52″ rotate and extend further distally, elongating the device between the head region 4 and the threads 52″. In this way, the break in the bone can be further reduced, and the proximal segment and distal segment of the bone can be pulled closer together. Opposite rotation (e.g., counter-clockwise) of the screwdriver results in relaxing the compression of the implantable device 42″. Continuing rotation (e.g., counter-clockwise) allows removal of the device.

FIG. 4C shows a stress-strain graph based on the implantable device 42 shown in FIG. 4B and FIG. B. The graph of elastic properties in FIG. 4C shows the effect of adding a deformable anchor 46 (as well as the helical cut 44 to the central zone 48) of a Nitinol device creating a spring like elastic action as both the deformable anchor 46 and helical cut 44 stretched and released (see the dotted line with right pointing and left pointing arrowheads showing, respectively, elastic stretch and elastic recovery). The dynamic compression working range 4e (between L4a and L4b) extends over 10%. As shown in arrow 4f in FIG. 4C, matching implant properties to bone properties with constant compression forces, greater than a 3.5% range of motion is achieved. The elastic match with human bone properties is shown. Installation of the implantable device into a bone requires no rotation or torque on the installer accessory and no tapping of the pre-drilled hole. The installed implantable device can be adjusted for position by rotation either in or out. The set-up also allows removal of the device if necessary, either immediately or later in time. As with any of the helical cut compression devices, the flexibility inherent with the helical cut shaft will improve fatigue failure resistance as well.

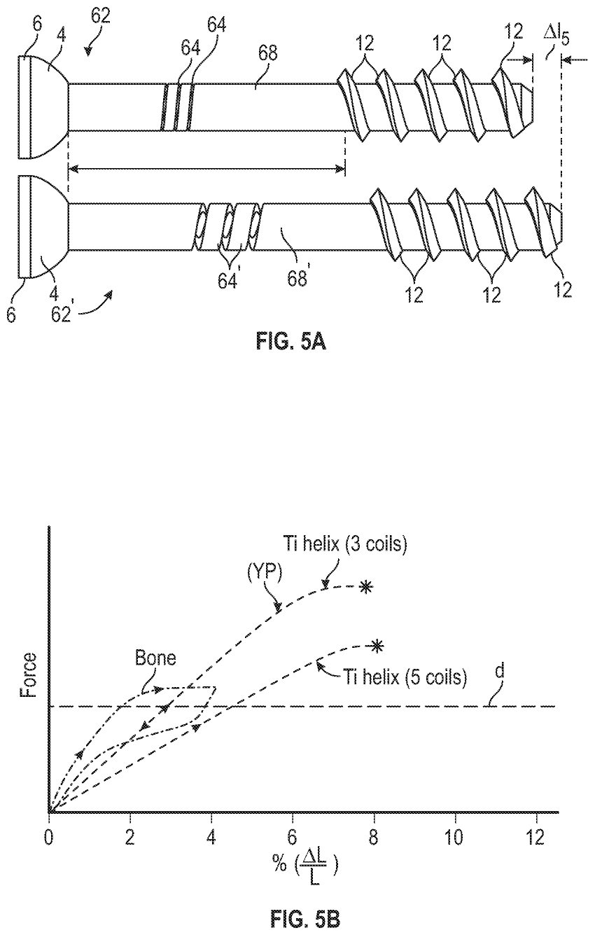

Also described herein are implantable non-Nitinol devices such as a cannulated titanium or stainless steel cannulated screw with a wide range of motion compression capability with the addition of a feature, such as a geometrical or mechanical feature, and the feature may control (at least in part) feature elasticity, feature compression, and/or feature length. The feature may be in a central zone of an implantable non-Nitinol device and different features may control and change device elasticity, device compression, and/or device length. In some examples, a feature may be a helical region or part of a helical region in the central zone of an implantable device. A feature, such as a helix (as used herein, a helix also includes a spiral and variations) may have spring or spring-like properties. A central zone (or an entire implant) may be made from a material that itself has limited range of compression (e.g., 0.2%), such as stainless steel or titanium implant material (e.g., β Ti or Ti64 (Ti6Al-4V) alloy. The central zone having the feature may be cannulated (hollow) or non-cannulated (e.g., a solid rod). In some examples, a feature may be a helical region or part of a helical region in the central zone of an implantable device. FIG. 5A shows an implantable device 62 and implantable device 62′. The implantable devices 62/62′ are similar to the implantable device 32 above except the implantable device 62 is not made from a substantially elastic material (e.g., Nitinol). The implantable device 62 is made from a substantially inelastic material, such as a titanium alloy or stainless steel. Titanium alloy or stainless steel devices on the market devices have a very limited compression range and virtually no matching or overlap of elastic properties with bone. Although the particulars of material properties of the titanium alloy or stainless steel of the implantable device 62/62′ is different from the material properties of Nitinol of the implantable device 32/32′, the a wide range of motion compression capability with the addition of a feature both have a wide and useful range of motion compression capability.

FIG. 5B shows a stress-strain curve of the implantable device 62/62′ shown in FIG. 5A with either 3 helical coils or 5 helical coils and a typical bone. FIG. 5B shows the desired compression level d. FIG. 5B also shows the yield point (YP) for an implantable device 62/62′ of titanium with 3 helical coils. The yield point for an implantable device 62/62′ of stainless steel with 3 helical coils is very similar. FIG. 5B illustrates the enhanced elastic effect of providing a helical cut pattern in the central zone of a titanium or stainless steel device. The effect is to allow this device acquire much enhanced elastic properties in compression range of motion. For example, from just 0.2% without the added helical cut to as much 6% or more total range of motion. Because titanium does not have the super elastic hysteresis behavior of Nitinol, this embodiment may not provide the constant force features of the devices of FIGS. 2A, 3A, and 4A. It also may not have as much of a range of motion, but it does offer significantly improved performance due to the matching bone characteristics.

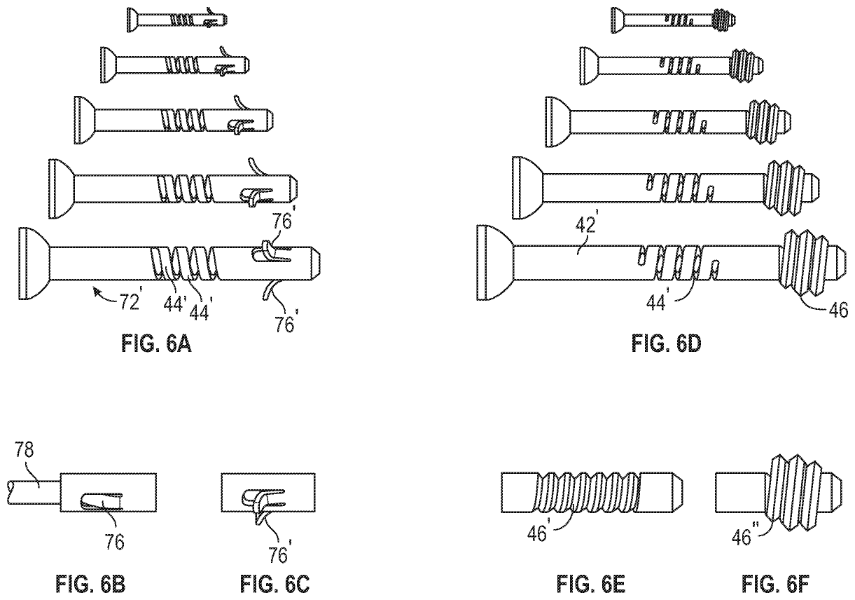

FIG. 6A shows different sizes of an implantable device 72′ with an expanded helical region 44′ and ready for use. These may be used for different types of bones or different types of injuries. The different sizes could be available in a kit for surgical use. The implantable device 72 is similar to the implantable device 32 except instead of threads on the distal end, the implantable device 72 has an anchor 76 including a plurality of tabs. The tabs can be in a compact configuration during insertion through the first bone segment and the second bone segment and take on an expanded configuration in the second bone segment. In some variations, the tabs are shape memory (e.g., Nitinol).

Also described herein is an insertion tool for inserting and installing an implantable device into a bone or other substrate. The insertion tool can hold the implantable device in a first, contracted configuration for insertion and then convert the implantable device to a second, expanded configuration. FIG. 6B and FIG. 6C show two enlarged fragmentary views showing the distal anchor region of one of the devices from FIG. 6A, shown first with the anchor 76 being held in a retracted state by the insertion tool 78 and then in a deployed state with the tool removed (FIG. 6C). The insertion tool and the implantable device may have mating parts (e.g., lock and key) or a frangible coupling between them. The insertion tool 78 can hold the implantable device 72 and anchor 76 in a first, contracted configuration (with the mating parts or frangible coupling) for insertion into a bone channel. The implantable device 72 and anchor 76 a second, expanded configuration shown in FIG. 6C. In some examples, inserting an implantable device into a bone or other substrate includes holding, with an insertion tool, the implantable device in a first, contracted configuration for insertion, inserting the implantable device through a bone, activating the insertion device to convert the implantable device to a second, expanded configuration.

FIG. 6D shows a shows different sizes of the implantable device 42 that could be used for different types of bones or different types of injuries. The different sizes could be available in a kit for surgical use. FIG. 6E and FIG. 6F are two enlarged fragmentary views showing the distal anchor region of one of the devices from FIG. 6C, shown first with helical threads in a retracted state (FIG. 6E) and then in a deployed state (FIG. 6F). The helical threads may be held in the retracted state with an insertion tool. Similar to as described above for FIG. 6B and FIG. 6C, some methods include the steps of holding, with an insertion tool, the implantable device in a first, contracted configuration for insertion, inserting the implantable device through a bone with the insertion tool, activating the insertion device to convert the implantable device to a second, expanded configuration, and releasing the implantable device from the insertion tool, and removing the insertion tool from the bone.

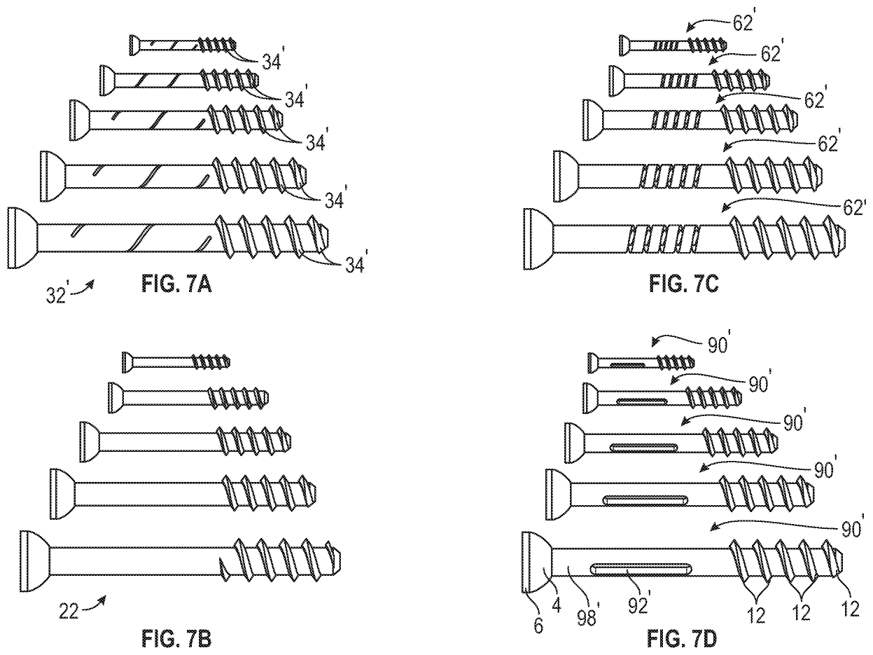

FIG. 7A shows different sizes of the implantable device 32′ that could be used for different types of bones or different types of injuries. The different sizes could be available in a kit for surgical use. FIG. 7B shows different sizes of the implantable device 22′ that could be used for different types of bones or different types of injuries. The different sizes could be available in a kit for surgical use. FIG. 7C shows different sizes of the implantable device 62′ that could be used for different types of bones or different types of injuries. The different sizes could be available in a kit for surgical use. FIG. 7D shows an implantable device 90′. The implantable device 90′ is similar to the implantable device 32′ except the implantable device 90′ has a longitudinal slot 92′ or cutaway region in the central zone 98′ instead of a helix 34′. The longitudinal slot 92′ or cutaway region increases the elasticity of the central zone 98′, better matching the material properties of the implantable device 90′ with bone. In some variations, the implantable device 90′ is made from Nitinol. In some variations, the implantable device 90′ is made from stainless steel or a titanium alloy.

Methods herein include securing bone segments (e.g., of a broken bone) together using dynamic compression configured to provide compression over a period of from a few hours to days, weeks, months, and/or years. Some methods include the step of introducing an implantable device through a first bone segment and at least partially into a second bone segment. Some methods include predrilling a first channel in the first bone segment and a second channel in the second bone segment. In some methods, the implantable device has an elongate body with a proximal end and a distal end; a head region at the proximal end and wherein the head region engages with a proximal end of the first bone segment (either directly or through a substrate such as a bone plate); a bone engagement part at the distal end wherein the bone engagement part engages with the second bone segment (e.g., an internal surface of a bone channel through the second bone segment); a dynamic compression portion between the head region and the bone engagement part, the dynamic compression portion in a first axially compact configuration.

Some embodiments include the step of transforming the dynamic compression portion into a second axially elongated configuration. Some embodiments include the step of urging the dynamic compression portion from the second elongated configuration towards the first, compact configuration, thereby urging the first bone segment and the second bone segment together. In some embodiments, the dynamic compression portion axially contracts by at least 0.5% relative to the length of the axially compact configuration. In some embodiments, the head region is wider than other portions of the elongate body. Some embodiments includes the step of drilling a first channel through the first bone segment and drilling a second channel at least partially through the second bone segment. In some embodiments, the implantable device is configured to axially contract at least 1%, at least 2%, at least 5% or at least 10% relative to the length of the axially compact configuration. Some embodiments include the step of contracting the implantable device by at least 1%, at least 2%, at least 5% or at least 10% relative to the length of the axially elongated configuration toward the axially compact configuration after the implantable device has been introduced into the bone segments. Some embodiments include the step of transforming the bone engagement part of the implant from a radially smaller structure to a radially larger structure and thereby engaging the second bone segment and holding the implantable device in the second bone segment. In some embodiments, the dynamic compression portion includes a hollow region having a helical slit through a wall thickness thereof. In some embodiments, urging includes axially compacting the helical slit. In some embodiments, a diameter of the dynamic compression portion remains relatively constant during the urging step. In some embodiments, the dynamic compression portion and bone engagement part comprise Nitinol. In some embodiments, the head region remains outside the first bone segment. In some embodiments, the head region remains outside the first bone segment. In some embodiments, the introducing step requires substantially zero insertion force (e.g., the implant may be placed or inserted into (pre-drilled) bone channels without requiring substantial pushing or torqueing on the part of the surgeon).

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable device comprising:
an elongate body with a proximal end and a distal end;
a head region at the proximal end;
a bone engagement part at the distal end configured to engage a bone;
a dynamic compression portion between the head region and the bone engagement part, the dynamic compression portion in either (i) a first axially compact configuration or (ii) a second axially elongated configuration, the dynamic compression portion comprising a material configured to transform between the first, compact configuration and the second elongated configuration,
wherein the second, elongated configuration is at least 0.5% longer than the first, compact configuration.

2. The implantable device of claim 1, wherein the dynamic compression portion comprises nitinol.

3. The implantable device of claim 1 wherein the dynamic compression portion comprises a cannulated rod.

4. The implantable device of claim 1, wherein the dynamic compression portion comprises a cannulated rod with a rod wall and a helical slit through a wall thickness thereof.

5. The implantable device of claim 1 wherein the dynamic compression portion comprises a helix having at least two, at least three, at least four, at least five, or at least six helical turns.

6. The implantable device of claim 1 wherein when the dynamic compression portion is in the second configuration the dynamic compression portion urges the proximal end and the distal end towards each other.

7. The implantable device of claim 1 wherein the second, elongated configuration is at least 1%, at least 2%, at least 5% or at least 10% longer than the first, compact configuration.

8. The implantable device of claim 1 wherein the bone engagement part comprises a screw thread.

9. The implantable device of claim 8 wherein the dynamic compression portion comprises a helix having at least two, at least three, at least four, at least five, or at least six helical turns and the screw thread and the helix turn in the same direction.

10. The implantable device of claim 8 wherein the dynamic compression portion comprises a helix having at least two, at least three, at least four, at least five, or at least six helical turns and the screw thread and the helix turn in opposite directions.

11. The implantable device of claim 1 wherein the bone engagement part comprises a helical thread.

12. The implantable device of claim 1 wherein the bone engagement part comprises a helical thread with either a first configuration with a smaller transverse width or a second configuration with a larger transverse width, wherein the helical thread is configured to transform between the first configuration and the second configuration in response to an applied or removed force.

13. The implantable device of claim 1 wherein the bone engagement part comprises an extendible tab.

14. The implantable device of claim 1 wherein bone engagement part has an outer diameter greater than an outer diameter of the dynamic compression portion.

15. The implantable device of claim 1 wherein the head region is wider than other portions of the elongate body.

16. A method of securing bone segments together comprising:
introducing an implantable device through a first bone segment and at least partially into a second bone segment, the implantable device having:
an elongate body with a proximal end and a distal end;
a head region at the proximal end wherein the head region is wider than other portions of the elongate body and wherein the head region engages with a proximal end of the first bone segment;
a bone engagement part at the distal end wherein the bone engagement part engages with the second bone segment;
a dynamic compression portion between the head region and the bone engagement part, the dynamic compression portion in a first axially compact configuration;
transforming the dynamic compression portion into a second axially elongated configuration; and
urging the dynamic compression portion from the second elongated configuration towards the first, compact configuration, thereby urging the first bone segment and the second bone segment together,
wherein the dynamic compression portion axially contracts by at least 0.5% relative to the length of the axially compact configuration.

17. The method of claim 16 further comprising drilling a first channel through the first bone segment and drilling a second channel at least partially through the second bone segment.

18. The method of claim 16 further wherein the implantable device is configured to axially contract at least 1%, at least 2%, at least 5% or at least 10% relative to the length of the axially compact configuration.

19. The method of claim 16 further comprising contracting the implantable device by at least 1%, at least 2%, at least 5% or at least 10% relative to the length of the axially elongated configuration toward the axially compact configuration after the implantable device has been introduced into the bone segments.

20. The method of claim 16 further comprising transforming the bone engagement part of the implant from a radially smaller structure to a radially larger structure and thereby engaging the second bone segment and holding the implantable device in the second bone segment.

21. The method of claim 16, wherein the dynamic compression portion comprises a hollow region having a helical slit through a wall thickness thereof.

22. The method of claim 16 wherein urging comprises axially compacting the helical slit.

23. The method of claim 16 wherein a diameter of the dynamic compression portion remains relatively constant during the urging step.

24. The method of claim 16 wherein the dynamic compression portion and bone engagement part comprise Nitinol.

25. The method of claim 16 wherein the head region remains outside the first bone segment.

26. The method of claim 16 wherein the introducing step requires substantially zero insertion force.
